(19) United States
(12) Patent Application Publication
(10) Pub. No.: US 2005/0177111 A1
(43) Pub. Date: Aug. 11, 2005

(54) Miniature infusion pump

(76) Inventors: Shaul Ozeri, Ramat Aviv (IL); Jehonatan Ozeri, Ramat Aviv (IL)

Correspondence Address: KATTEN MUCHIN ROSENMAN LLP, 575 MADISON AVENUE, NEW YORK, NY, 10022-2585, US

(21) Appl. No.: 10/773,672

(22) Filed: Feb. 6, 2004

(51) Int. Cl. A61M 37/00

(52) U.S. Cl. 604/154

(57) ABSTRACT

A micro-pump that supplies medication from a reservoir to a patient through a flexible tube is disclosed. The pump, which is especially suitable but not confined to delivering insulin to diabetic patients, is small and light enough to be attached to the body using adhesive plaster or to be strapped to the body in any other manner. In the preferred embodiment the pump mechanism comprises a lead screw, a weak force rotating element, an actuator, and a high force holding element. The device also includes a processing circuitry for controlling and monitoring the drive mechanism, a force sensor to measure medication pressure which generates indicative signals to the processing circuitry, a sensor for tracking the position of the syringe plunger, and a remote control unit.

### BACKGROUND OF THE INVENTION

The present invention relates to the field of infusion pumps for controlled delivery of medication to patients, more specifically to an infusion pump with an improved minute lightweight drive mechanism.

Infusion pumps deliver a volumetrically controlled medication to the patient over a period of time. A processing circuitry controls the periodic delivery of dosages of medication to a patient at predetermined rates. Infusion pumps often contain an electrical motor which rotates a lead-screw; the rotation of the lead-screw causes a nut to linearly move along it. The nut pushes a plunger through a syringe or a cartridge internal to the pump that causes medication to move from the syringe to the patient along the infusion path.

Prior art of Infusion pumps contain a large electrical motor which are strong enough to rotate the lead-screw against the opposing pressure of the medication inside the syringe. Such mechanism is described, for example, in U.S. Pat. Nos. 6,248,093, 5,637,095, 5,097,122, and 5,505,709. These devices contain electrical motors which are relatively large and heavy. Since dosages are given at discrete intervals over a period of time, each time the processing circuit activates the motor it consumes large current to operate.

The in addition to the disadvantages in size, weight and power consumption of existing medication pumps, theses devices also suffer from an additional drawback which stems from the principles according to which they operate. The amount of medication delivered from the device into the patient's body is controlled by the operation of the motor. The accuracy of these devices is therefore hard to control and dependent on the reliability and accuracy of the operation of the motor; minute fluctuations in the motor's behavior might cause significant deviations in the amount of medication delivered to the patient. The medication delivery is therefore calculated statistically.

As solutions to this problem elaborated devices have been developed to detect and respond to inconsistent flow rates. In cases of pressure buildup inside the syringe most commonly these devices compensate for the reduction of flow by changing the time intervals between successive pulses while informing the user of that change. If the pressure reaches the occlusion level, the pump stops pumping and the user is alerted. Due to the limitation of the motor, this is not a very satisfactory solution. Further, once the blockage is opened, the pressure which is built inside the container and delivery tube is released through the tube, forcing a possibility dangerously larger than prescribed dose of medicine into the patient's body.

There is therefore a need for a medication pump that in addition to being very small, lightweight and low in energy consumption will be able to deliver accurate and consistent dosage rate of medication over periods of time.

### SUMMARY OF THE INVENTION

A micro pump device for dispensing proportioned quantities of medical fluid. The medical fluid which may be, but is not limited to, insulin, is driven into the patient's body by applying pulsed pressure on syringe's plunger stem containing the medical fluid. The medical fluid is injected through syringe-tube connector to the patient's body. The device comprised of the following components: a vertically expending actuator means for applying pressure at the direction of the syringe plunger wherein the actuator activation is controlled by a programmable logic means; a stopper element for preventing the actuator movement in the opposite direction; plunger stem holder for preventing the plunger's movement back toward the actuator; guiding walls for applying pressure on the plunger stem holder; power mechanism causing gradual movement of the stopper toward the actuator. The actuator may be a piezoelectric (PZT) element which expends in the direction of the plunger stem upon receiving electrical current; another option is that the actuator is an electromagnetic actuator.

According to the first embodiment the power mechanism is an electric motor which is controlled by programmable logic means and the stopper element is then a nut lever connected to a screwing nut which is screwed along a lead screw, said lead screw is rotated by the electric motor.

According to the second embodiment the power mechanism is a spring and the stopper mechanism is comprised of two cylinders elements, connected by a supporting spring, which apply pressure on the guiding walls and are connected by a second actuator. The second actuator contracts in reaction to electric pulses, pulling the cylinder elements toward each other, decreasing the pressure on the guiding walls and enabling the spring to pull the stopper element toward the actuator. The given pulses are controlled by programmable logic means. The second actuator may be a Shape Memory Alloy (SMA) actuator.

The operation of the device is controlled by programmable logic means. The said logic means is a microprocessor controller which coordinates the operation of the power means and of the actuator in accordance with predefined parameters determined by the user. The controller further alerts the user of malfunctions.

The controller receives feedback about the operation of the device from two sources: an optical linear encoder and a force sensor resistor. The optical linear encoder gives indications as for the position of the stopper mechanism; the force sensor resistor measures changes in the movement of the plunger and the pressure within the syringe. These measurements are compared against defined plan values and analyzed to give an accurate report of the status of operation.

The device's housing is small, lightweight and watertight. The device may include a remote control unit for the user's control interface.

The stopper, which can be manually adjusted to its initial position for the purpose of reloading the syringe, may be comprised of a split nut to allow of adjusting the stopper to its initial position.

### BRIEF DESCRIPTION OF THE DRAWINGS

These and further features and advantages of the invention will become more clearly understood in the light of the ensuing description of a preferred embodiment thereof, given by way of example only, with reference to the accompanying drawings, wherein

FIG. 1 is a schematic overview of the first embodiment of the present invention;

FIG. 1A portrays a detailed illustration of the physical mechanism and a block diagram of the logical mechanism of first embodiment of the present invention;

FIG. 2 is a detailed illustration of the mechanism of the first embodiment of the present invention;

FIG. 2A illustrates the infusion pump's drive mechanism according to the first embodiment of the present invention in its initial position;

FIG. 2B illustrates the second stage of the operation cycle of the infusion pump's drive mechanism according to the first embodiment of the present invention;

FIG. 2C illustrates the third stage of the operation cycle of the infusion pump's drive mechanism according to the first embodiment of the present;

FIG. 2D illustrates the final stage of the operation cycle the infusion pump's drive mechanism according to the first embodiment of the present invention;

FIG. 3 is a detailed illustration of the mechanism of the second embodiment of the present invention;

FIG. 3A illustrates the infusion pump's drive mechanism according to the second embodiment of the present invention in its initial position;

FIG. 3B illustrates the second stage of the operation cycle of the infusion pump's drive mechanism according to the second embodiment of the present invention;

FIG. 3C illustrates the third stage of the operation cycle of the infusion pump's drive mechanism according to the second embodiment of the present;

FIG. 3D illustrates the fourth stage of the operation cycle of the infusion pump's drive mechanism according to the second embodiment of the present;

FIG. 3E illustrates the final stage of the operation cycle the infusion pump's drive mechanism according to the second embodiment of the present invention;

FIG. 4 illustrates the configuration of the stop mechanism of the second embodiment;

FIG. 5 illustrates the syringe loading operation according to the second embodiment of the present invention.

### DETAILED DESCRIPTION OF THE PREFERRED EMBODIMENTS

The present invention discloses a new small lightweight mechanism for a controlled drug infusion to a patient. The mechanism is integrated in a miniature apparatus which operates on low energy and may dispense precise quantities of chemical reagents into a patient's body, having improved dynamic range of operation.

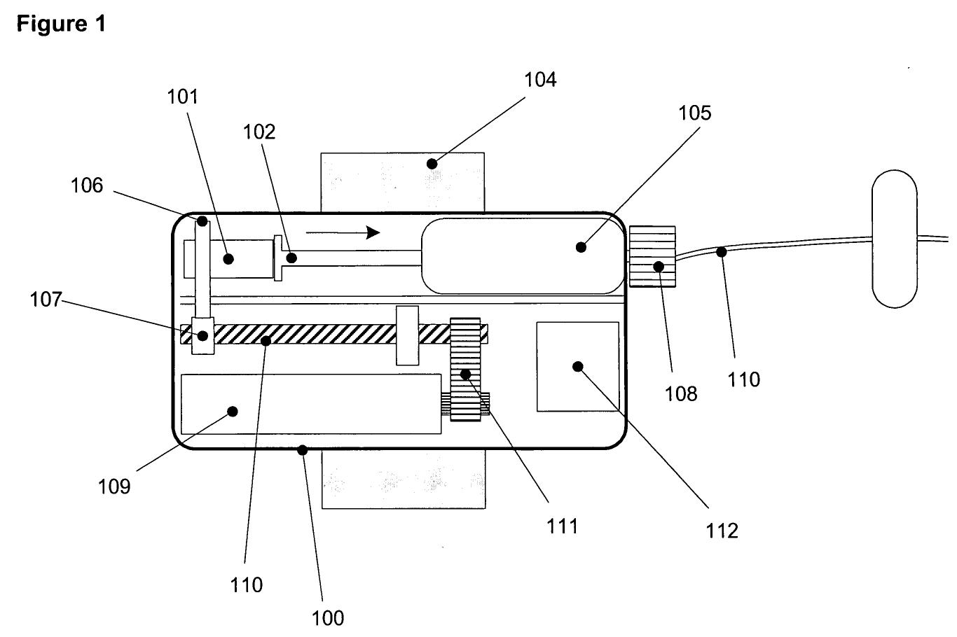

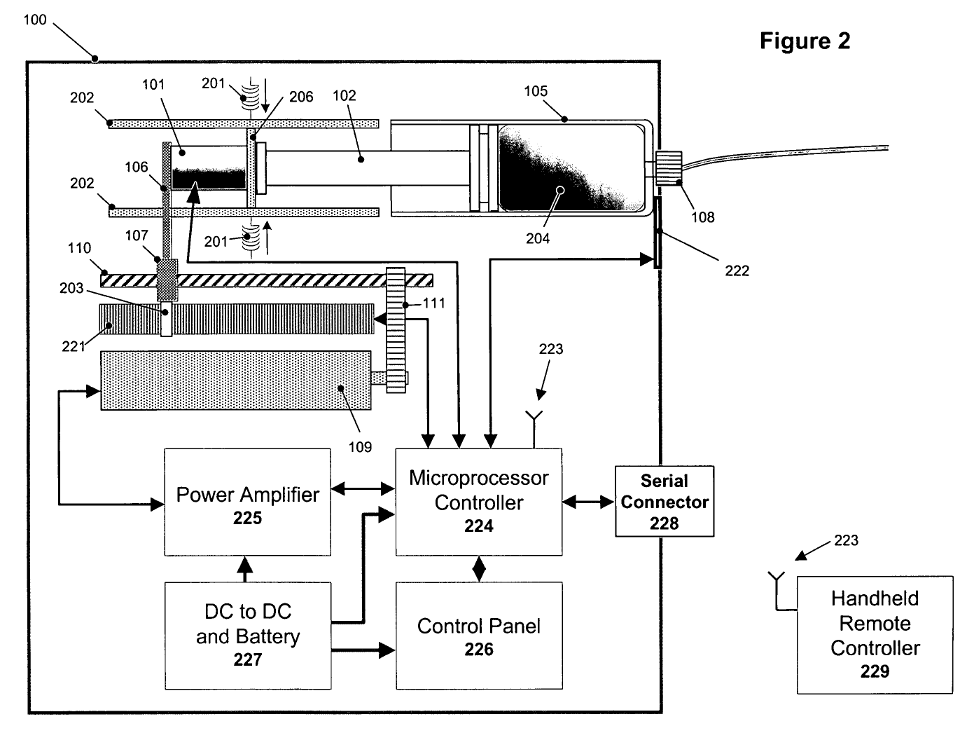

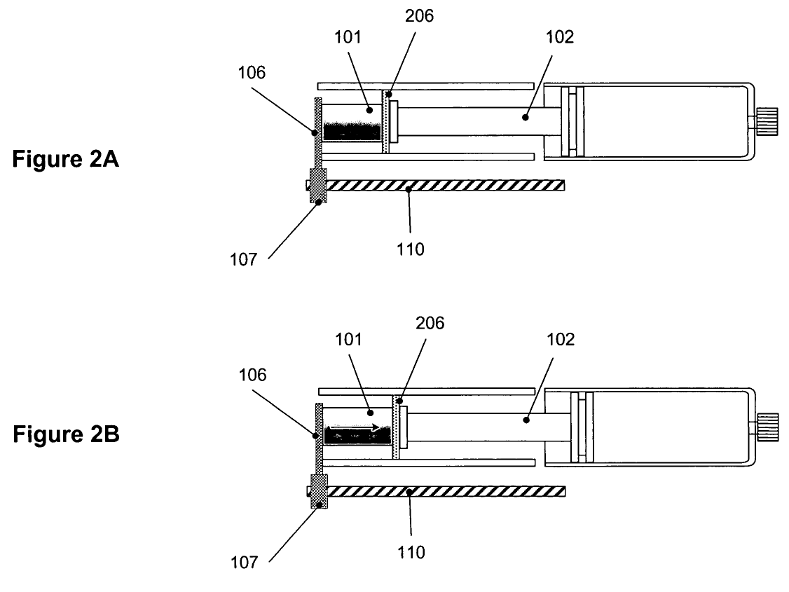

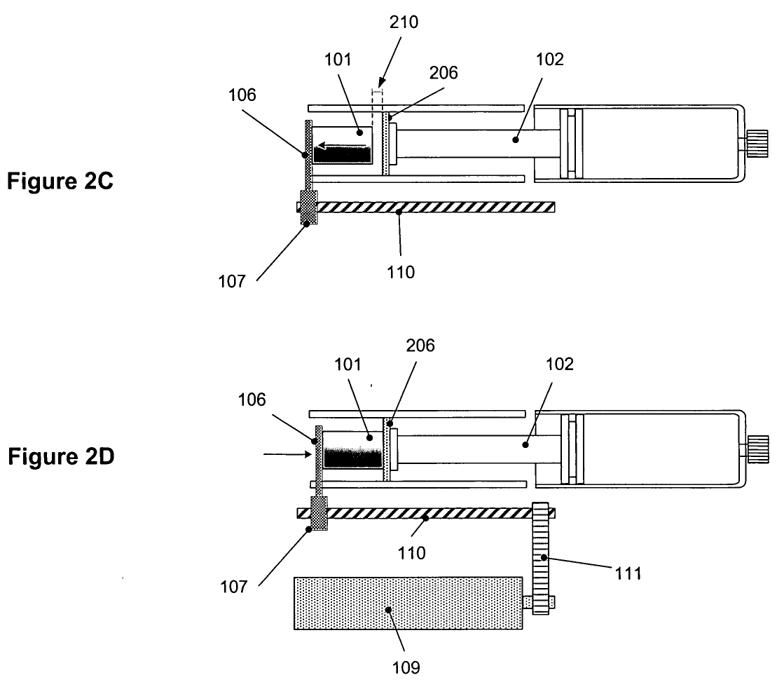

The general structure of the apparatus is illustrated in FIG. 1. The apparatus is composed of a waterproof device container 100, a syringe-tube connector 108, a tube 110 and attachment means 104 that fasten the device's container 100 to the patient's body or to a belt. As illustrated in FIG. 2, the container 100 is comprised of a mechanism for driving the chemical reagent, which is in the syringe's hollow barrel 204, through the syringe-tube connector 108 to the tube 110 that leads to the patient's body.

The chemical reagent is slowly released from the syringe 105 as a controlled amount of pressure is applied on the syringe's hollow barrel 204 by the plunger stem 102. The pressure of the plunger stem 102 is created by the expansion of the piezoelectric (PZT) actuator 101 which is placed at the plunger stem's 102 opposite end. Between the plunger 102 and the PZT actuator 101 there is a plunger stem holder 206 that is in contact with the guiding walls 202. There is a substantial friction at these points of contact that ensures that the plunger may move forward towards the syringe 105 whenever a pressure pulse is applied in that direction, but prevents it from moving back in the direction of the PZT actuator 101 when pressure stops. The pressure on the plunger stem holder 206 is stabilized by the guiding walls springs 201, or by an internal spring inside the plunger stem holder 206. PZT actuators 101 convert electrical energy to mechanical energy by expending in analog proportions to the voltage that is applied to them. In order to produce a controllable amount of pressure on the syringe's hollow barrel 204, an electric current is applied to the PZT actuator 101. The actuator is situated between the plunger stem 102 and a stopper 106 which prevents it from expending in the opposite direction. The full change in length of the PZT actuator is therefore applied on the plunger stem 102. The stopper 106, which in the present embodiment is a nut lever, is connected to a screw nut 107 that is screwed on a lead screw 110. The lead screw 110 is connected to an electrical motor 109 through a belt transmission 111 or via a gear mechanism (not shown). Due to this connection, the rotary motion of the motor 109 causes a similar motion of the lead screw 110. The mechanism is adjusted so that the motion of the motor 109 causes the screw nut 107 and the nut lever 106, which operate as a stopper, to move forward towards the syringe.

The four steps of this mechanism's cycle of operation are illustrated in FIGS. 2A-2D. FIG. 2A illustrates the mechanism at the initial stage of each cycle. In FIG. 2B an electric current is applied to the PZT actuator 101 which causes it to expend in the direction of the plunger stem 102. Having expended, the current to the PZT actuator 101 is turned off and the PZT actuator 101 shrinks back to its normal size as illustrated in FIG. 2C. The plunger stem holder 206 then holds the plunger stem 102 in place, and prevents it from moving back to its initial position such as in FIG. 2A. A gap 210 is them created between the PZT actuator 101 and the plunger stem holder 206. This gap is completely reduced by the operation of the motor 109 as illustrated in FIG. 2D. Through the belt transmission 111 or the gear mechanism the motor 109 turns the lead screw 110 which causes the screw nut 107 and the nut lever 106 to move forward and reduce this gap 210.

The operation of the device is manages, synchronizes and monitors by the microprocessor controller 224. It controls the activation of the PTZ actuator 101 and the motor 109 according to parameters given by the user and coordinates between them. It also receives feedback indications for the operation of the device, such as from the optical linear encoder 221 and from the Force Sense Resistor (FSR) 222 for example. The optical linear encoder 221 is a linear array of photodiodes. Attaching a led 203 to the screw nut 107 enables the encoder 221 to detect changes in the position of the screw nut 107 and the nut lever 106 in a high level of resolution. This is used to monitor the movement of the plunger, to verify it is moving according the preprogrammed scheme and has not been mechanically stuck. The FSR 222 is a resistor which changes its electrical resistance according to the mechanical pressure on it. It is placed between the syringe 105 and the device's container 100 and is used to measure the fluid pressure and to warn the controller 224 whenever sudden changes of pressure occur in a state of occlusion. As an alternative the FSR 222 may be placed between the plunger stem holder 206 and the plunger stem 102 or between the PZT actuator 101 and the plunger stem holder 206. Inconsistencies in the amount of pressure measured by the FSR 222 are then calculated accordingly. A different method for finding the exact location of the plunger stem holder 206 and amount of pressure in the syringe hollow barrel 204 at any given moment utilizes the transition of a high frequency ultrasound signal. The signal transmitter is attached to the PTZ actuator 101 and is transmitted in the direction of the syringe. In this case a mirror is placed at the far end of the syringe and the returning signal may give indications as for this distance or for the amount of pressure inside the syringe hollow barrel 204.

The device also includes a control panel 226 which operates as the user interface and allows determining the manner in which the device operates. Through the control panel 226 users may turn the device on and off and control the dosages of chemical reagents released by the device over any period of time. The control panel 226 also provides the users with indicators of the device's mode of operation: whether it is operating normally or if there is any abnormality in its operation. Since the device may be attached directly to the patient's body, the present invention may also include a hand held wireless remote control 229 to facilitate the controlling procedures of the device. The remote control 229 establishes a wireless communication with the controller 224 via the wireless link 223. The device's includes a source of energy 227, such as a battery, taking into account that the operation of the mechanism consumes low levels of energy, and that the device is intended to be as small and as lightweight as possible. Also included is a power amplifier 225 that ensures that the motor 109 receives ample power for operation and a serial connector 228 for uploading and downloading data.

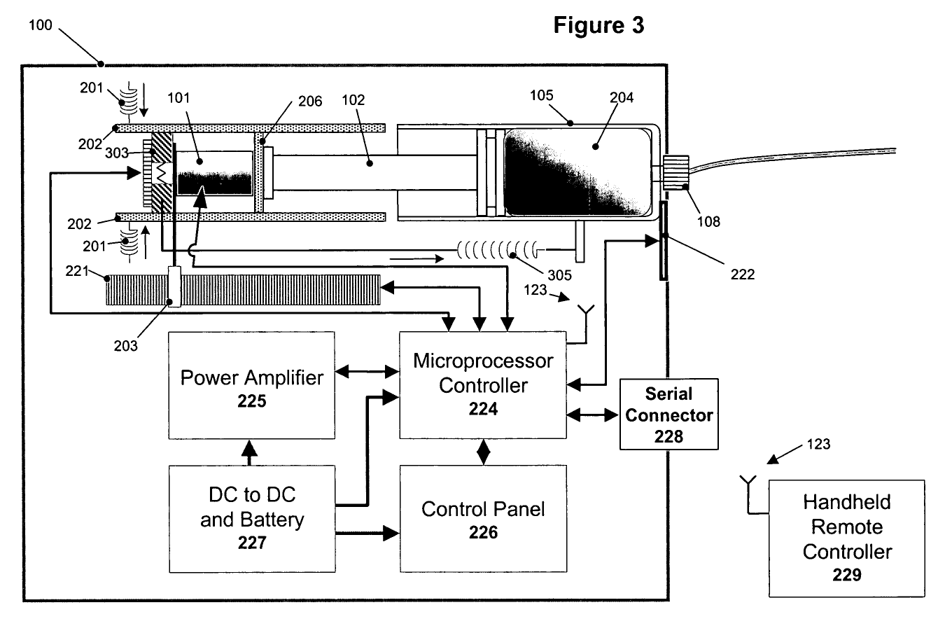

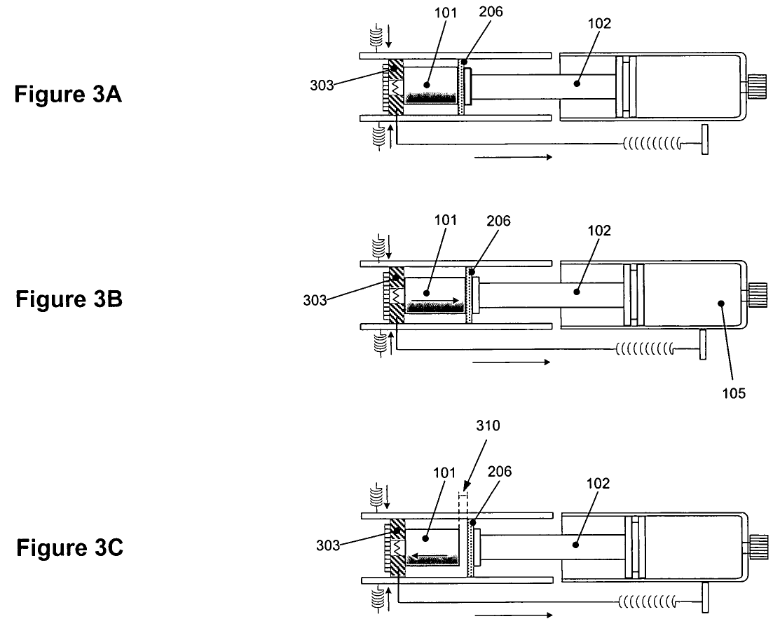

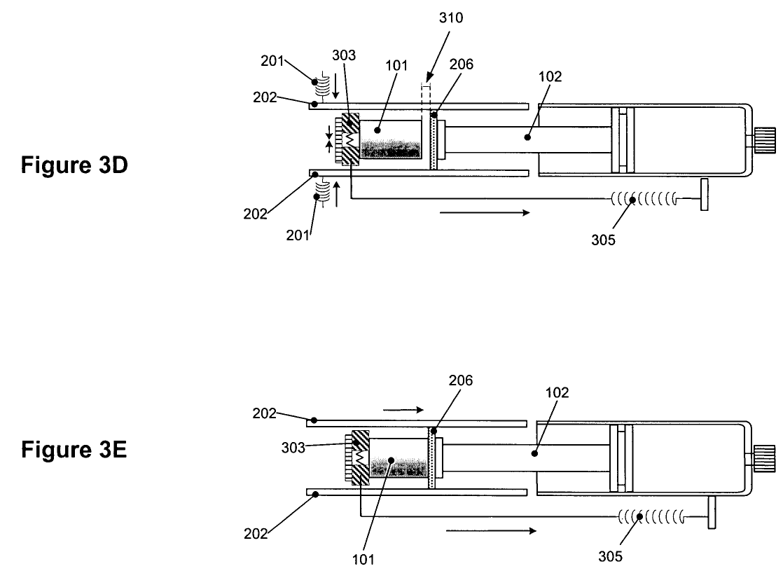

The second embodiment of the said mechanism is illustrated in FIGS. 3-7. As illustrated in FIG. 3 the structure of the second embodiment is in many ways similar to the one of the first embodiment. It utilizes a different stopper mechanism 303 from one in the first embodiment 106 and therefore does not include a motor 109 and its accompanying mechanism (the belt transmission 111 or gear, the lead screw 110, the screw nut 107 and the nut lever 106). The stopper mechanism 303 of the second embodiment is described in details below.

The five steps of the cycle of operation of the second embodiment are illustrated in FIGS. 3A-3E. The operation of the plunger stem 102, the plunger stem holder 206 and the PZT actuator 101 is identical to the one described in the first embodiment, but instead of a nut lever 106 operating as a stopper at the far end of the PZT actuator 101, there is a different stopper mechanism 303 whose constitution is described below. FIG. 3A illustrates the initial position of the mechanism in the cycle of operation. In the second stage, which is illustrated in FIG. 3B, the PTZ actuator 101 receives an electrical current and expends in the direction of the plunger stem 102 and the syringe 105. The other end of the PTZ actuator 101 is connected to a stopper 303, which prevents it from expending in the opposite direction. In the third stage in FIG. 3C the electrical current is stopped and the PTZ actuator 101 shrinks to its normal size. A gap 310 is then created between the PTZ actuator 101 and the plunger stem holder 206. At the fourth stage of the cycle of operation, which is portrayed in FIG. 3D, the stopper 303 detaches from the mechanism's guiding walls 202, according to a method that is described below. And finally, in the fifth stage described in FIG. 3E the main pulling spring 305 pulls the PTZ actuator 101 in the direction of the plunger stem holder 206 and reduces gap 310 completely.

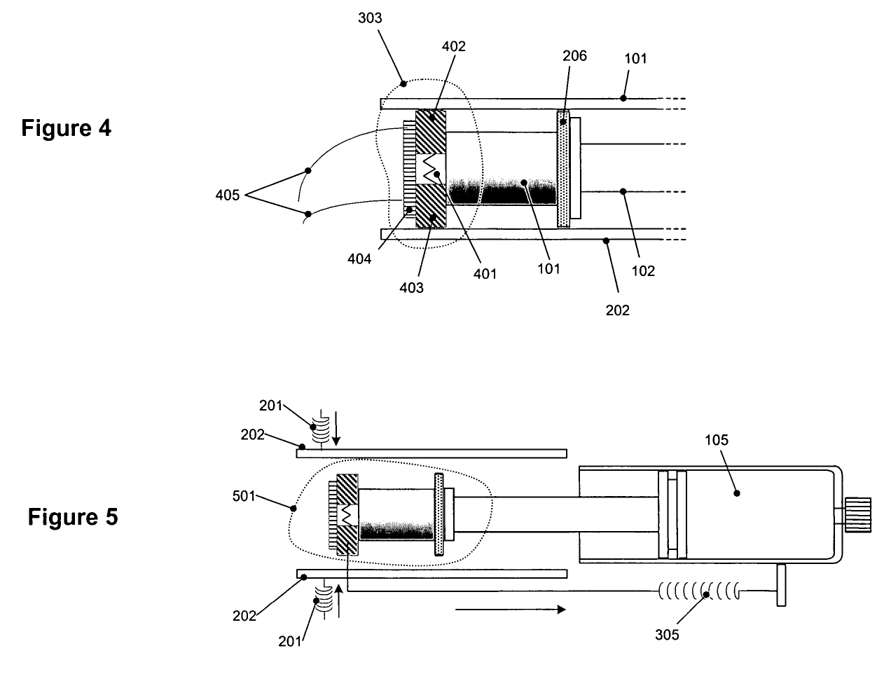

The constitution of the stopper 303 and its method of operation are illustrated in FIG. 4. The stopper 303 comprises of an upper holding cylinder 402 and a lower holding cylinder 403 which are connected by a cylinder internal spring 401 and a shape memory alloy (SMA) actuator 404. The spring 401 allows the two cylinders 402, 403 to reduce and expend their relative proximity and allows flexibility in the amount of pressure that the cylinders 402, 403 apply on the guiding walls 202. The SMA actuator 404 contracts in length when electrically heated and easily returns to its normal size as it cools back to an ambient temperature. In the normal state of the stopper 303 the cylinders 402, 403 are pressed against the guiding walls 202 and resist movement whenever the PZT actuator 101 expends and ensure that the full length of the PZT actuator's 101 expansion is in the direction of the plunger 102. After the PZT actuator 101 contracted back to its normal size the SMA actuator 404 receives an electric current via wires 405 and heats up. The change in temperature causes the SMA actuator 404 to contract. As the SMA actuator 404 contracts it pulls the upper and the lower cylinders 402, 403 towards each other, reduces the pressure at their points of contact with the guiding walls 202 and enables the stopper 303 and the PZT actuator 101 to move freely along the guiding walls 202. This operation enables the fourth and fifth steps of the second embodiment.

Similarly to the first embodiment the operation of the second embodiment is managed by a microprocessor controller 224, powered by a source of energy such as a battery 227, programmed through a control panel 226 and a hand held remote control. The monitoring mechanism of the second embodiment also operates in the same manner as the one of the first embodiment by utilizing a Force Sense Resistor (FSR) 222 and an optical linear encoder 221. The only difference is that instead of being attached to the screw nut 107 the led 203 in this embodiment may be attached to a rod that is connected to the stopper 303 to enable the encoder 221 to detect changes its position, monitor the movement of the plunger, verify that it is moving according the preprogrammed scheme and has not been mechanically stuck.

The manual operation of loading the syringe according to the first embodiment includes the following steps: first, the guiding walls 202, which are held in place by springs 201, are released by the user and then the nut lever 106 is released from the screw nut 107. The user may them return the screw nut 107 back into its initial position at the far end of the lead screw 110 and the nut lever 106 can then reconnect to the screw nut 107. Returning the screw nut 107 to its initial position may be achieved by screwing it back on the lead screw 110, or by using a split nut mechanism which allows for easily changing the position of the screw nut 107 on the lead screw 110. The user removes the syringe 105, fills it with the appropriate chemical reagents and places it back into place in the device. The device is then ready to be turned on and put into use.

The manual operation of loading the syringe according to the second invention is illustrated in FIG. 5. The guiding walls 202, which are held in place by springs 201 or by clips, are released by the user and the plunger pushing mechanism 501 is then free to be pulled back into its initial position. As in the loading of the syringe according the first embodiment, the user removes the syringe 105, fills it with the appropriate chemical reagents and places it back into place in the device. Once the user puts the guiding walls back in their initial position the device is ready to be turned on and put into use.

In the embodiments described above the PTZ actuator 101 may be replaced by a SMA or by electromagnetic solenoid without having to change any of the other components of the invention.

While the above description contains many specifities, these should not be construed as limitations on the scope of the invention, but rather as exemplifications of the preferred embodiments. Those skilled in the art will envision other possible variations that are within its scope. Accordingly, the scope of the invention should be determined not by the embodiment illustrated, but by the appended claims and their legal equivalents.

1. A micro pump device for dispensing proportioned quantities of medical fluid by applying pulsed pressure on syringe's plunger stem containing the medical fluid which is injected through syringe-tube connector to the patient's body, said device comprised of:
a vertically expending actuator means for applying pressure at the direction of the syringe plunger wherein the actuator activation is controlled by a programmable logic means;
a stopper element for preventing the actuator movement in the opposite direction;
plunger stem holder for preventing the plunger's movement back toward the actuator;
guiding walls for applying pressure on the plunger stem holder;
power mechanism causing gradual movement of the stopper toward the actuator.

2. The device of claim 1 wherein the power mechanism is a mechanical spring.

3. The device of claim 1 wherein the actuator is piezoelectric element which expends in the direction of the plunger stem upon receiving electrical current.

4. The device of claim 2 wherein the actuator is an electromagnetic actuator.

5. The device of claim 2 wherein the power mechanism is an electric motor which is controlled by programmable logic means and the stopper element is a nut lever connected to a screwing nut which is screwed along a lead screw, said lead screw is rotated by the electric motor.

6. The device of claim 2 further comprising a second actuator, wherein the stopper element is comprised of two cylinders elements which apply pressure on the guiding walls and are connected by a second actuator, wherein said second actuator contracts in reaction to given pulses, pulling the cylinder elements toward each other, decreasing the pressure on the guiding walls and enabling the spring to pull the stopper element toward the actuator, wherein the given pulses are controlled by programmable logic means.

7. The device of claim 4 wherein the second actuator is a Shape Memory Alloy actuator

8. The device of claim 4 wherein the stopper further comprises an internal spring wherein the internal spring is situated in between the cylindrical elements applying pressure thereof.

9. The device of claim 1 further comprising supporting springs applying outside pressure on the guiding walls.

10. The device of claim 1 wherein the programmable logic means is a microprocessor controller which coordinates the operation of the power means and of the actuator in accordance with predefined parameters determined by the user and further alerts the user of malfunctions.

11. The device of claim 1 further comprising optical linear encoder and force sensor resistor for measuring changes in the movement of the plunger and the pressure within the syringe wherein said measurements are compared against defined plan values and analyzed for providing the controller input data as for monitoring the operation status.

12. The device of claim 1 wherein the device housing is watertight.

13. The device of claim 1 further comprising communication means for communicating with a remote control unit.

14. The device of claim 1 wherein the medical fluid is insulin.

15. The device of claim 1 wherein the stopper can be manually adjusted to its initial position.

16. The device of claim 5 further comprises a split nut for purpose of adjusting the stopper to its initial position.
